(19) United States
(12) Patent Application Publication
(10) Pub. No.: US 2002/0025314 A1
(43) Pub. Date: Feb. 28, 2002

(54) Homeopathic compositions for the treatment of viral and metabolic deseases

(76) Inventor: Marie-Christine Etienne, Tassin La Demi Lune (FR)

Correspondence Address: Richard E. Fichter, BACON & THOMAS, PLLC, Fourth Floor, 625 Slaters Lane, Alexandria, VA, 22314, US

(21) Appl. No.: 09/839,366

(22) Filed: Apr. 23, 2001

Related U.S. Application Data
(63) Continuation-in-part of application No. 09/239,698, filed on Jan. 29, 1999, now abandoned.
Application No. 09/239,698 is a continuation of application No. 08/381,884, filed on May 9, 1995, now abandoned.
Application No. 08/381,884 is a 371 of international application No. PCT/FR92/00813, filed on Aug. 21, 1992.

(51) Int. Cl. A61K 39/395; A61K 33/00

(52) U.S. Cl. 424/131.1; 424/600

(57) ABSTRACT

A method for causing the elimination of an active principle, R, from cells of a mammal which contain the active principle, R, which comprises administering to said mammal, a compound identical in nature to said active principle in a homeopathic product of the formula RxCH, in which R is the active principle and in which xCH is a homeopathic dilution of said active ingredient R to eliminate R from the cells to restore normal function to the perturbed pericellular transport systems.

### CROSS-REFERENCE TO RELATED APPLICATIONS

This application is a Continuation-in-part of co-pending application Ser. No. 09/239,698, filed Jan. 29, 1999 (of which the entire disclosure of the pending, prior application is hereby incorporated by reference), which is a continuation of application Ser. No. 08/831,884, filed May 9, 1995, and which application is a 371 of PCT/FR92/00813, filed Aug. 21, 1992.

### BACKGROUND OF THE INVENTION

In the prior art, the therapeutic use of homeopathic remedies consists in causing a series of symptoms to disappear in a diseased individual by administering a homeopathic dilution of the substance which has been able to cause the same symptoms in a healthy subject.

### SUMMARY OF THE INVENTION

The present invention concerns the use of a homeopathic product for the preparation of a medicament intended to bring about the elimination from the cells which contain it of a compound that is identical in nature to the active principle in the specific case in which said elimination result in restoring the normal operation of the pericellular transport systems which are disturbed.

### DETAILED DESCRIPTION

The present invention relates the use of a homeopathic product for the preparation of a drug intended for treating metabolic diseases.

The general formula of the homeopathic product is RxCH.

The metabolic diseases to which the invention relates are diseases characterized by the intracellular accumulation or intracellular deficit of a chemical substance of simple or complex formula , which can vary from case to case and is designated here by R.

In fact, R acts on the pericellular transport systems with respect to itself, which systems have broken down and to which it restores correct function. These diseases are frequently referred to as genetic in the prior art.

The xCH dilutions are defined as follows: 15 to 30 CH for diseases caused by intracellular retention. 4CH, 5CH 7CH, 9CH for diseases caused by an intracellular deficit.

For some of these metabolic diseases, the pathological manifestations are the direct consequence of the intracellular accumulation or intracellular deficit of a chemical substance. For others (of these diseases ) the relationship with the intracellular deficit or excess is very indirect: The consequence of this excess or deficit is the production by the cell of abnormal substances responsible for the observed pathological manifestations. These substances have a structure possessing anomalies which are due to the abnormal conditions in which the cell functions. The cells start to produce antibodies of abnormal structure which are responsible for allergies or, in the case of autoantibodies responsible for autoimmune diseases. Thus secondary to the anomalies of the transport systems, the cells start to produce all kinds of chemicals of abnormal structure or in abnormal amounts which are responsible for a very wide variety of complaints such as hypertension in the case of an overproduction of aldosterone gout in the case of an overproduction of uric acid.

The invention assert that all diseases caused by the intracellular deficit or excess of a chemical substance, and qualified in the prior art as genetic, are due to dysfunctions of pericellular transport systems with respect to the chemical substance which is in excess or deficit inside the cells.

The invention, which is based on these novel and original theories is based on the use of the property of elimination of chemical substances from the cell and from the organism under the influence of their homeopathic dilution in order to restore normal function to the perturbed pericellular transport systems with respect to these chemical substances. In the invention, the ion channels and pericellular transport systems function along concentration gradients which cause the ions and other substances to pass from the more concentrated medium to the less concentrated medium. According to the laws of osmosis, water follows the particles and it is the medium containing the larger number of molecules which becomes the less concentrated medium, so the transport systems become blocked in the direction of entry into the cell if the cell already contains an excess of the chemical substance in question, or in the direction of exit from the cell if the cell already contains a deficit of the chemical substance in question.

These transport systems behave like a multitude of doors operating in only one direction, cf. the entrance doors of a supermarket, which allow entry or exit. These doors which allow the chemical substance in question to pass are blocked either in the direction of entry into the cell when there is an intracellular excess of this chemical substance, or in the direction of exit from the cell when there is an intracellular deficit of this chemical substance.

In the invention, in the case of diseases caused by intracellular retention, for example of minerals, water passes into the cells, where the minerals are already present in large amounts, and reduces the concentration inside the cell, where there is the greatest amount of minerals. (Minerals are taken as a nonlimiting example, but it is possible to have intracellular retention of other products of various chemical types.) The minerals therefore have an indefinite tendency to accumulate inside the cell, where they are already in excess. The property of elimination of intracellular components from the cell and from the organism under the influence of their homeopathic dilution is immediately applicable to this type of complaint. The homeopathic dilution of the component in excess causes the component (or whatever the chemical substance may be) which is in excess to leave the cells, reducing the intracellular overloading. However, there is another mechanism to explain an action on the pericellular transport systems: the administration of the homeopathic dilution of the product which is in excess in the cell also causes water to be drawn around the eliminated molecules, since water follows the minerals and other products and leaves the cell. The extracellular medium becomes punctually less concentrated than the intracellular medium. The pericellular transport systems corresponding to this point, which are regulated along a concentration gradient, i.e. some of the entrance doors swing to expel from the cell the product which could only enter it. The consequences of these punctual and repeated eliminations of a few molecules are a long-term restoration of normal function to the transport systems under the influence of the repeated administration of the homeopathic remedy in question over weeks, months and years. The dilutions used according to the invention are preferably high, for example 15CH to 30CH, because, according to the invention, these high dilutions cause a more vigorous elimination than low dilutions. Their action extends over several consecutive days, so their frequency of administration is 1 or 2 times a week.

In the invention, in diseases caused by an intracellular deficit of any product, for example a mineral, the deficient intracellular medium impoverished in molecules of ions or of another product contains only a little water, since the water has flowed towards the extracellular sector rich in mineral ions (or another product). The interior of the cell has become more concentrated than the exterior of the cell, and the transport systems, which are regulated along a concentration gradient and transport from the more concentrated medium to the less concentrated medium, are blocked in the exit direction.

In the invention, the administration in this case of a homeopathic dilution of the product which is in intracellular deficit causes:

either the elimination from the cell of the few remaining molecules of the product or mineral, which causes the intracellular concentration to drop punctually to zero and thereby incites a transporter to swing in the direction of entry in order to cause the deficient ion or product to enter the cell at the point where the concentration was zero;

or neutralizes molecules of the product or mineral in the extracellular sector and eliminates them to leave the water molecules which alone surrounded them. According to the laws of osmosis, to re-establish an equal water concentration on either side of the cell membrane, these water molecules enter the cell and it is the cell which will punctually become the less concentrated medium. The doors of the corresponding transport system swing and position themselves in the direction of entry into the cell.

In the invention, repeated administrations of a homeopathic dilution of the mineral or product which is in intracellular deficit have the long-term consequence of swinging a large number of molecular transport systems in the direction of entry into the cells and restoring normal function to these cells by restoring normal function to the pericellular transport systems. The dilutions used 'in the invention are preferably low, non-limiting examples being 4CH, 5CH, 7CH or 9CH, because these dilutions cause a less vigorous elimination than high dilutions. Their effect is short-lived and they must be administered daily in the invention.

In the invention, the restoration of normal function to the transport systems cures metabolic diseases and anomalies which are secondary to these dysfunctions but in which the relationship with the transport systems and the intracellular deficits or excesses can be obvious and direct or can be very indirect and not at all obvious.

In the invention, the posology of the homeopathic dilution is as follows: the administration of the homeopathic dilution of the product in question must be regular and can last for a long time, often for several years.

A) Diseases Caused by an Intracellular Excess:

### EXAMPLE 1

### Cystic Fibrosis

Cystic fibrosis is a so-called genetic disease characterized by an excess of sodium chloride in the secretions and inside the cells. Some authors also report an excess of potassium. No treatment exists in the prior art. Researches are based on the hypothesis that it would be possible to use a protein called CFRT, which would prevent NaCl from entering the cells, or that it would be possible to use genetic manipulations. The object of the invention is to treat cystic fibrosis with a compound of the formula RxCH in which R, by way of indication and without implying a limitation, is NaCl or any other salt capable of acting on the ion overload in question and the defective ion channel, such as potassium chloride, sodium iodide or iodine chloride, or compound salts of sodium, chlorine, potassium and iodine which comprise all or some of these elements,

### EXAMPLE 2

### Pigmentary Retinopathy

Pigmentary retinopathy is an incurable so-called genetic disease which is due to an accumulation of melanotic or other pigments in the cells of the retina and which culminates in blindness. The invention consists in treating this complaint with the compound RxCH in which R, by way of indication, is melanin or Sepia, a well-known homeopathic active product which is melanin mixed with impurities, or another pigment,

### EXAMPLE 3

### Oxalosis

This is a so-called genetic disease in which there is an intracellular accumulation of oxalic acid or calcium oxalate. This complaint is observed in children and culminates in death due to renal insufficiency. The invention consists in treating oxalosis with the compound RxCH in which R is oxalic acid or calcium oxalate

### EXAMPLE 4

### Hyperkalemic Periodic Paralysis

This is a so-called genetic disease which consists of attacks of paralysis caused by an intracellular as well as extracellular excess of potassium. Its treatment in the prior art consists in administering diuretics which inhibit carbonate dehydratase. Geneticists locate the genetic anomaly on chromosome 17. The object of the invention is to treat this disease with the compound RxCH in which R is potassium or a potassium salt for example dipotassium phosphate (or Kalium, Phosphoricum, a known homeopathic active product)

### EXAMPLE 5

### Hemochromatosis

Hemochromatosis is a so-called genetic disease due to the intracellular accumulation of iron. Its treatment in the prior art consists of blood lettings and the administration of Desferal, a chelating agent for iron. The object of the present invention is to treat hemochromatosis with the compound RxCH in which R is iron.

### EXAMPLE 6

### Wilson's Disease

Wilson's disease is a disease due to the intracellular accumulation of copper. Its treatment in the prior art consists in administering penicillamine, a chelating agent for copper. The object of the present invention is to treat Wilson's disease with the compound RxCH in which R is copper.

### EXAMPLE 7

### Alzheimer's Disease

This is a disease in which an excess of aluminum has been found. The invention relates to the use of the compound RxCH to treat Alzheimer's disease. R is aluminum or 1 other aluminum salt or derivative

B) Diseases Caused by an Intracellular Deficit:

### EXAMPLE 8

### Tetany

This is a disease which consists of muscular spasms caused by an intracellular deficit of calcium or magnesium. In the case of an intracellular deficit of magnesium, this disease is said to be genetic and its treatment in the prior art consists in administering magnesium. The invention in this case relates to the use of the compound RxCH in which R is dimagnesium phosphate (Magnesia Phosphorica) to treat the so-called genetic form of tetany.

In other cases of tetany, R is tricalcium phosphate, Calcarea Phosphorica, or other salts or chemical substances.

### EXAMPLE 9

### Vitamin-resistant Rickets

This is a particular form of rickets in children. The present invention consists in treating this complaint with the compound RxCH in which R is:

calcium or mineral salts derived from calcium Calcarea carbonica

oyster limestone or

tricalcium phosphate.

### EXAMPLE 10

### Hypokalemic Periodic Paralysis

This is a so-called genetic disease which, like Example 4, consists of attacks of paralysis, except that the paralysis here is caused by an intracellular as well as extracellular deficit of potassium; the object of the invention is to treat it with the compound RxCH in which R, by way of indication, is potassium or 1 potassium salt, for example dipotassium phosphate or Kalium Phosphoricum, a well-known homeopathic active product,

### EXAMPLE 11

### Anemia Caused by an Intracellular Deficit of Iron

This is a little-known complaint which is characterized in that the red blood corpuscles are small and the hernatocrit is low, and which manifests itself by symptoms of anemia. The invention consists in treating this form of anemia with the compound RxCH in which R is iron

C) The Group of Diseases which are Related that Vary Secondarily to the Transport-system Anomalies which Caused Them:

### EXAMPLE 12

### Rheumatoid Polyarthritis

The clinical studies performed by the inventor have shown that a variant of this autoimmune disease of previously indeterminate origin is due to an anomaly of the pericellular transport systems with respect to black antimony sulfide, there being an intracellular deficit of black antimony sulfide. The object of the invention is to treat this complaint with the compound RxCH in which R is black antimony sulfide for juvenile chronic polyarthritis. For adult rheumatoid polyarthritis: Flouric acid and graphites.

The invention further relates to the treatment of other autoimmune complaints, not accompanied by polyarthritis, with the same compound RxCH in which R is preferably black antimony sulfide. The inventor has observed a case of antiplatelet autoinimunization which falls in this category.

### EXAMPLE 13

### Systemic Lupus Erythernatosus

The invention relates to the treatment of a variant of this autoinimune complaint with the compound RxCH in which R is gold.

### EXAMPLE 14

### Amyotrophic Lateral Sclerosis

The clinical studies performed by the inventor have shown that this incurable disease may be an autoimmune disease caused by an anomaly of the pericellular transport systems with respect to phosphorus. The invention allow the obtention of a drug for treating this disease with the compound RxCH in which R is phosphorus or a salt derived from phosphorus.

Multiple sclerosis is a very similar disease which benefits from the same type of treatment with the same compound RxCH in which R is a salt derived from phosphorus such as calcarea phosphorica with another salt: causticum.

### EXAMPLE 15

### Hyperthyroidism

The clinical studies performed by the inventor have shown that a variant of this disease is due to a disorder of the pericellular transport systems with respect to NaC1, there being an intracellular deficit of NaCl. It will be noted that cystic fibrosis, which involves an intracellular excess of NaCl, is a totally different entity. The invention allows the obtention of a drug for treating hyperthyroidism with the compound RxCH in which R is NaCl.

D) The Group of Miscellaneous Diseases:

### EXAMPLE 17

### Miscellaneous Diseases

Finally, the following complaints may be mentioned as other examples of metabolic diseases, without implying a limitation: Refsum's disease, or R can be phytanic acid, Charcot-Marie-'Tooth and Dejerine-Sottas disease, Huntington's chorea, where R can be zinc, Thevenard's disease, Friedrich's disease, Pierre Marie's hereditary cerebellar ataxia, Strumpell Lorrain's periodic paralysis, Roussy-Levy syndrome, dyslipidosis, idiopathic mental retardations in children, and autism.

Some clinical observations of cases of patients treated with compounds RxCH belonging to a medicament which may be obtained by the invention are reported below.

CASE No. 1

A 72-year-old female patient presenting with a very matt complexion and slight trembling of the two upper limbs. A systematic examination for an episode of asthenia shows abnormally high serum iron at 173 microg/liter (μ/l), i.e. 31 micromol (μmol), on October 11, but not hemochromatosis. Ferrum metallicum. 15CH is prescribed at a rate of one dose every Sunday for a period of one month starting on October 17.

On November 16, i.e. at the end of the treatment, the serum iron is assayed again. It has fallen to 103.41 microg (μg), i.e. 18.50 Micromol (μmol). The patient has suffered no hemorrhaging and the very significant drop in the serum iron from

31 micromol (μg/l) to 18.5 micromol (μmol)

173 microg/l (μ/l) to 103 microg/l (μ/l) is due to the administration of Ferrum metallicum, which has caused the elimination of the iron from the organism. The general condition is improved. The trembling is less pronounced.

A further assay of the serum iron is requested 2½ months later at the end of January, i.e. 2½ months after cessation of the one-month treatment with Ferrum metallicum 15CH. The serum iron has risen but has not reached its original level. It is:

25 (micromol/l(μmol/l), i.e. 139.75 microg/l (μ/l), proving that the patient carries a metabolic anomaly which gives her a spontaneously high iron level, or that she absorbs iron abnormally, which is not the case since her diet excludes this possibility. On January 31st, Ferrum metallicum 15CH is prescribed again at a rate of one dose every Sunday for one month with no repeat assay at the end of the treatment. During this winter, an improvement in the general condition is noted, together with an absence of the bronchitis episode which usually occurred every winter and lasted about one month. The following July, the serum iron is assayed again and is:

26.80 Micromol/l (μmol/l), i.e. 149.81 microg/l (μg/l), in other words steady.

This case illustrates the theories and experiments pertaining to guinea-pigs, namely that the product whose homeopathic dilution has been administered is eliminated from the organism. This elimination involves a withdrawal of ions from, the cells, the iron being stored intracellularly. Furthermore, the patient cited here, who has a spontaneous tendency towards high serum iron, could well carry a minor and very attenuated heterozygous form of hemochromatosis, and this case would also concern the theories of the invention pertaining to this complaint.

CASE No. 2

A 44-year-old female patient presenting with hyperthyroidism due to multinodular thyroid hyperplasia, and morning allergic rhinitis. Mapping showed hypertrophy of the right lobe with a cold nodule. The biological examinations performed after the first consultation showed hyperthyroidism with the following results:

| | | |
|---|---|---|
| on 19 February | TSH: | 0.02 |
| | T4f: | 35 |

The normal levels are 0.2 to 4 for TSH and 10 to 25 for T4f. The TSH is abnormally low and the free T4 abnormally high. The patient receives treatment with a synthetic thyroid inhibitor, namely Basdene 9 on its own, starting on February 22, at increasing doses of 1 tbl on the 1st day, 2 tbl on the 2nd day and then 3 tbl per day. on 3 March TSH: 0.02

| | | |
|---|---|---|
| on 3 March | TSH: | 0.02 |
| | T4f: | 36.8 |

Therefore, a few days after the start of the Basdene ® treatment, the hyperthyroidism has remained identical and even become slightly worse. On March 7, the patient is seen again by the consultant and, in place of Basdene®, Neo-mercazole® is prescribed at doses of 3 tbl 3 times a day:

| | | |
|---|---|---|
| on 17 March | TSH: | 0.02 |
| | T4f: | 33.8 |

The thyroid hormone level after 10 days of Neomercazole is still just as high, with no improvement.

On March, 20 the patient is seen again and the following is added to the Neo-mercazole treatment for the allergic rhinitis:

Natrium muriaticum 6DH, 3 tbl/1 for 7 days, then

Natrium muriaticum 4CH, 3 granules/1 for 15 days.

On April 13 T4f. 25.1

For the first time, the thyroid hormone level has normalized again and the abundance of the allergic rhinitis has reduced by ¾.

In order to know whether it is the Natrium muriaticum or the Neomercazole 9 which has normalized the thyroid functions, the Neo-mercazole® is suddenly withdrawn, which, if it is the Neo-mercazole® which has lowered the T4f, should have the effect of raising it immediately to 30, and the regular administration of Natrium muriaticum 4CH is continued on April 13.

On April 27: T4f=26.9, i.e. still normal, proving that it is indeed the Natrium muriaticum and not the Neo-mercazole® which has cured the hyperthyroidism and normalized the level of T4f. The precise doses of Natrium muriaticum which were in fact taken are:

Natrium muriaticum 6DH: 3tbl/day from March 20 to April 11, then Natrium muriaticum 4CH: 3 granules/day from April 11 to April 28. On April 28, the patient is seen again by the consultant and the prescribed dose of Natrium muriaticum is further modified, being increased to Natrium muriaticum 5CH: 3 granules a day. The Neo-mercazole® was of course never restarted. The effect of this change in doses of Natrium muriaticum is to further lower the thyroid hormone:

| | | |
|---|---|---|
| on 21 April | T4f: | 26.9 |
| on 10 June | T4f: | 18.6 |

The following dose of Natrium Muriaticum 5CH is still continued from this time on: 3 granules a day.

On August 1, the allergic rhinitis is found to have disappeared.

Meanwhile, on July 10, the patient had an operation on her multinodular hyperplastic goiter containing cold nodules, which was suspected of neoplasia despite the numerous punctures which had never shown anything alarming. There was no cancer on the operated part. On August 1 the result of this operation is a T4f of 13.8, which is now near the lower limit of the norm (the norm being 10 to 25). A tendency towards hypothyroidism might be feared. This is not so at all:

| | | |
|---|---|---|
| - on 9 October | TSH: | 2.14 |
| | T4f - | 16.7 |

The thyroid functions are therefore normal. The allergic rhinitis has disappeared, the Natrium muriaticum 5CH still being continued at a rate of 3 granules a day. In conclusion, this example illustrates the fact that hyperthyroidism is indeed a disease caused by an intracellular deficit of NaC1, and that the prescription of NaCl 5CH, by acting on the restoration of normal function to the transport systems, virtually immediately normalizes the thyroid hormone metabolisms which were perturbed.

Another perturbed metabolism normalizes again: this is the production of abnormal type 1 gE antibodies responsible for the allergic rhinitis, which ceases completely after a few months of treatment and which had begun to decrease and improve as soon as the first doses of homeopathic remedy had been administered.

CASE No. 3

A 7-year-old female child suffering from juvenile chronic polyarthritis, an autoimmune disease qualified as genetic.

After examination of the clinical case, it appears that the homeopathic symptoms correspond to an intracellular deficit of black antimony sulfide. At the first consultation, the patient presents with polyarticular symptoms localized especially in the elbows and knees, requiring a daily administration of Naprosyn® and physiotherapy sessions. The Naprosyn® is taken at a dose of 1 tbl of 250 mg a day and is not sufficient to suppress the symptoms, since the disease worsens. It is this which motivates consultation. Antimonium crudum 5CH, 3 to 5 granules once a day, is added to the Naprosyn® as from December 27. The articular condition improves so substantially after this date that the doses of Naprosyn®, which would have had to be increased, are reduced; at the beginning of the following April, the Naprosyn® is completely withdrawn and a small amount of Voltaren® is taken from time to time. The following July, the right arm, which was deformed, has completely straightened again. The elbow joint has normalized again. The symptoms of polyarthritis have disappeared. Of course, the child took 3 to 5 granules of Antimonium crudum 5CH every day throughout this time. As from July, the antiinflammatories are withdrawn and the treatment with Antimonium crudum 5CH is still continued. It will be maintained for several years.

Conclusion

This case illustrates the fact that it has been possible to cure a so-called genetic autoimmune disease by restoring normal function to the pericellular transport systems.

Chronic rheumatoid polyarthritis here proves to be a disease caused by an intracellular deficit of black antimony sulfide.

In conclusion, this case and the previous case verify the theory developed invention, which asserts that correction of the functional anomalies of the pericellular transport systems secondarily normalizes the perturbed metabolisms.

CASE No. 4

A 3½-year-old Alsatian-type dog suffering from canine autoimmune polyarthritis, a disease which would correspond to systemic lupus erythematosus in human pathology. The symptoms of this complaint are complete seizure of the hindquarters and a fever of 40° or even 41°. The conventional medical treatment starts on Jul. 8, 1986 and consists in injecting corticoids at a rate of:

Solu-Medrol® 20 mg morning and evening, IM, under a blanket of antibiotics maintained for about ten days. The corticoids are then gradually reduced. The Solu-Medrol® is replaced with Cortancyl® 5 mg: 2 tbl a day.

The homeopathic symptoms corresponded to an intracellular deficit of gold and to the prescription of Aurum metallicum. In August of the same year, a homeopathic remedy is added to the Cortancyl®. However, the wrong homeopathic remedy is given: instead of Aurum metallicum, the dog is given Mercurius solubilis: 3 to 5 granules a day. As from the beginning of September, trusting in a cure, the Cortancyl® is gradually reduced. The disease immediately recurs and it is necessary to resume the treatment with Solu-Medrol® 20 mg morning and evening, 1M, for about ten days, again followed by Cortancyl® 5 mg: 2 tbl/day, the Mercurius solubilis of course being withdrawn.

On March 18th of the following year, when it has been possible to reduce the doses of Cortancyl® 5 mg to 1 tbl/day, Aurum metallicum 5CH, 3 to 5 granules a day, which is now the night prescription, is added this time. Two months later, it has been possible successfully to reduce the Cortancyl®, which is now taken at a dose of only 1 tbl of 5 mg once a week, every Monday. For a few days in April, the administration of Aurum metallicum 5CH is forgotten, which, one morning, causes aphonia (complete loss of voice and bark) and by slight incipient stiffness of the hindquarters. The treatment with Aurum metallicum 5CH is immediately resumed at a rate of 3 granules several times a day (3 times), accompanied by Ledum plaster 7CH: 3 granules twice a day, for 3 days. The treatment is subsequently continued at the following doses:

Aurum Metallic 5CH: 5 granules a day

Aurum metallicum 7CH: 3 granules a day

The last ½ tablet of Cortancyl® 5 mg was given on Sep. 28, 1987 and the corticoids were then definitively withdrawn. The homeopathic treatment was continued at the above-indicated doses until August 1989, when it was definitively withdrawn. No relapse ever occurred.

Conclusion

This case illustrates and confirms the theories of the invention and shows that this form of canine autoimmune polyarthritis is due to an intracellular deficit of gold and that the corresponding human disease, systemic lupus erythematosus, is probably also due to an intra-cellular deficit of gold. It also shows that a treatment with a medicament which may be obtained by the invention in the long term restores normal function to the pericellular transport systems without the possibility of a relapse, and that restoring this normal function to the transport systems suppresses the metabolic anomalies of the cells, i.e. in this case the production of abnormal antibodies.

CASE No. 5

A 20-year-old female patient who presented with several malaises characteristic of tetany. The diagnosis is in fact spasmophilia, namely tetany caused by a deficit of magnesium and not calcium, since her calcium level is normal and only her erythrocyte magnesium level is lowered, the serum magnesium level being normal; this anomaly has been described as a genetic disease characterized 'by a solely iantracellular deficit of magnesium. With the framework of the present invention, it is considered that the ion channel pertaining to magnesium has broken down and no longer allows magnesium to enter in the normal amount for the cell's needs. The object of the present invention is to make magnesium enter the cells in the normal amount again. The assays prior to treatment were carried out on 2 occasions:

for the 1st time in December 1985, when the disorders started:

calcium: 102 mg (N: 93 to 107)

erythrocyte magnesium: 32 mg (N: 35 to 40)

serum magnesium: 20 mg (N: 19 to 23)

the second time in 1991, when the treatment started:

serum magnesium: 0.84 mmol/l (N: 0.75 to 0.95)

erythrocyte magnesium: 2.26 mmol/l (N: 2.30 to 2.65)

The treatment consisted in administering Magnesia Phosphorica 6DH: 3 tablets 3 times a week. The tetany attacks disappeared from the start of the treatment. After 3 months, a further assay shows that only the erythrocyte magnesium has risen to a normal level, the serum magnesium level having dropped slightly; this proves that the magnesium did indeed enter the cells and that this magnesium which entered was indeed the serum magnesium.

serum magnesium: 0.73 mmol/l (N: 0.75 to 0.95)

erythrocyte magnesium: 2.38 mmol/l (N: 2.30 to 2.65)

After 2½ years of treatment, this good result was maintained in both clinical and biological terms with the following levels:

serum magnesium: 0.97 mmol/l (N: 0.75 to 0.95)

erythrocyte magnesium: 2.20 mmol./l (N: 1.85 to 2.30)

Before this last analysis, in addition to the treatment with Magnesia Phosphorica, magnesium had been administered in the form of trace elements; this had not been prescribed before and explains the large increase in serum magnesium.

This observation clearly illustrates the specific action on the intracellular ion concentrations by action on the ion channels: measurement of the erythrocyte magnesium is the only biological assay which currently makes it possible to assess an ion concentration inside a cell, namely the red blood corpuscle, and hence to assess what is happening inside all the other cells of the organism.

CASE No. 6

A 28-year-old female patient who presented with symptoms of colitis together with various pathological manifestations such as heavy legs, warts, fatigue and some forms of lipothymia with symptoms of tetany.

Systematic serum and erythrocyte magnesium assays on this patient show a deficit only of erythrocyte magnesium:

serum magnesium: 23 mg (N: 18 to 24)

erythrocyte magnesium: 41 mg (N: 45 to 55)

A treatment based on Magnesia Phosphorica 4CH and then 5CH does not raise the erythrocyte magnesium level. On the other hand, a one-month treatment with Magnesia Muriatica 7CH raises the erythrocyte magnesium level very substantially to give the following assays:

serum magnesium: 18 mg (N: 18 to 24)

erythrocyte magnesium: 52 mg (N: 45 to 55)

which clearly illustrates the very high specificity of the mineral salt on the corresponding ion channel:

Magnesia Phosphorica. is dimagnesium phosphate,

Magnesia Muriatica is magnesium chloride.

The normal values are indicated in brackets since these values change according to the laboratory performing the analysis. One of the laboratories even changed its calibration, and consequently the values it indicated as normal, between 2 assays performed a few months apart.

What is claimed is:

1. A method for causing the elimination of an active principle, R, from cells of a mammal which contain the active principle, R, which comprises administering to said mammal, a compound identical in nature to said active principle in a homeopathic product of the formula RxCH, in which R is the active principle and in which xCH is a homeopathic dilution of said active ingredient R to eliminate R from the cells to restore normal function to the perturbed pericellular transport systems

2. The method of claim 1, characterized in that the compound to be eliminated is a mineral compound and that the effect of its elimination is to restore normal function to an ion channel.

3. The method of claim 1, characterized in that the compound to be eliminated is a poison or part of a poison.

4. The method according to claim 1 which is for treating diseases caused by a metabolic error consisting either of an intracellular accumulation of a compound of simple or complex formula, or of an intracellular deficit of such a compound.

5. The method according to claim 4, characterized in that the consequence of the metabolic error is the production of abnormal metabolises, especially abnormal antibodies such as autoantibodies.

6. The method according to claim 4, characterized in that the metabolic error is genetically determined.

7. The method according to claim 5, characterized in that the metabolic error is genetically determined.

8. The method of claim 1, wherein xCH is hahnemannian or korsakowian homeopathic dilution.

9. The method of claim 1, wherein x is 4, 5, 7, 15 or 30.

10. The method of claim 9, wherein the active principle to be eliminated is a mineral compound and normal function is restored to an ion channel.

11. The method of claim 11, wherein x is 4, 5, 7, 15 or 30 and the active principle to be eliminated is a poison or part of a poison.

12. The method of claim 1, wherein the active principle to be eliminated is a virus or a viral particle.

13. A method of treating a disease caused by metabolic error consisting either or an intracellular accumulation of a compound of simple or complex formula, or of an intracellular deficit of such a compound which comprises administering to cells containing such a compound R or in need of said compound R, a homeopathic product having a formula RxCH in which R is said compound and in which xCH is its homeopathic dilution to treat said disease caused by metabolic error consisting either of an intracellular accumulation of said compound of simple or complex formula, or of an intracellular deficit of such a compound.

14. The method of claim 13, characterized in that the consequence of the metabolic error is the production of abnormal metabolites.

15. The method of claim 14, wherein the abnormal metabolites are abnormal antibodies.

16. The method of claim 15, wherein the abnormal antibodies are autoantibodies.

17. The method of claim 16, wherein the metabolic error is genetically determined.

18. The method of claim 16, wherein x is 4, 5, 7, 15 or 30.

19. The method of claim 2, wherein the mineral compound is a potassium, calcium or antimony containing mineral compound.

20. The method of claim 2, wherein R is a mineral compound which is a potassium, calcium or antimony containing mineral compound.

21. Application of homeopathic compounds of the general formula RxCH to the elimination of an intracellular chemical substance R from the cell, when this elimination causes the restoration of normal function to the perturbed pericellular transport systems, in order to obtain drugs which, by causing this elimination from the cell are intended for restoring normal function to the perturbed pericellular transport systems.

22. The application of claim 21, wherein xCH is hahnemannian or korsakowian homeopathic dilution, R is a mineral compound which is a potassium, calcium or antimony containing mineral compound and wherein x is 4, 5, 7, 15 or 30.
